(19) United States
(12) Patent Application Publication
(10) Pub. No.: US 2022/0392587 A1
(43) Pub. Date: Dec. 8, 2022

(54) METHODS AND SYSTEMS FOR DETERMINING ONE OR MORE STANDARDIZED BILLING CODES ASSOCIATED WITH AN EXAMINATION OF A PATIENT

(71) Applicant: Cowell IP Company LLC, Virginia Beach, VA (US)

(72) Inventor: Justin D. Cowell, Virginia Beach, VA (US)

(21) Appl. No.: 17/336,656

(22) Filed: Jun. 2, 2021

(51) Int. Cl. G16H 10/60; G16H 80/00; G06N 20/00

(52) U.S. Cl. CPC G16H 10/60; G16H 80/00; G06N 20/00

(57) ABSTRACT

The present disclosure is directed to determining one or more standardized billing codes associated with an examination of a patient. In particular, the methods and systems of the present disclosure may: receive data generated based at least in part on one or more notations of a medical provider with respect to an examination of a patient; receive data generated based at least in part on one or more interactions between the patient and physical infrastructure of a medical organization associated with the medical provider; and determine, based at least in part on one or more machine learning (ML) models, the data generated based at least in part on the notation(s), and the data generated based at least in part on the interaction(s) between the patient and the physical infrastructure of the medical organization, one or more standardized billing codes associated with the examination of the patient.

### FIELD

The present disclosure relates generally to medical billing. More particularly, the present disclosure relates to methods and systems for determining one or more standardized billing codes associated with an examination of a patient.

### BACKGROUND

Healthcare has been, and is projected to continue to be, one of the fastest growing economic sectors. Numerous private and public entities fund healthcare through collected premiums and taxes, as well as public and private financing. Healthcare billing is often readily susceptible to error and fraud. For example, providers may intentionally or unintentionally incorrectly code and/or upcode healthcare products and/or services, as well as inappropriately bundle such products, services, and/or the like. Patients, providers, insurers, governments, and the public at large have vested interests in ensuring proper coding, billing, reimbursement, and/or the like for healthcare products, services, and/or the like.

### SUMMARY

Aspects and advantages of embodiments of the present disclosure will be set forth in part in the following description, or may be learned from the description, or may be learned through practice of the embodiments.

One example aspect of the present disclosure is directed to a method. The method may include receiving, by one or more computing devices, data generated based at least in part on one or more notations of a medical provider with respect to an examination of a patient. The method may also include receiving, by the computing device(s), data generated based at least in part on one or more interactions between the patient and physical infrastructure of a medical organization associated with the medical provider. The method may further include determining, by the computing device(s) and based at least in part on the data generated based at least in part on the notation(s) of the medical provider and the data generated based at least in part on the interaction(s) between the patient and the physical infrastructure of the medical organization, one or more standardized billing codes associated with the examination of the patient.

Another example aspect of the present disclosure is directed to a system. The system may include one or more processors and a memory storing instructions that when executed by the processor(s) cause the system to perform operations. The operations may include receiving data generated based at least in part on one or more notations of a medical provider with respect to an examination of a patient. The operations may also include receiving data generated based at least in part on one or more interactions between the patient and physical infrastructure of a medical organization associated with the medical provider. The operations may further include determining, based at least in part on one or more machine learning (ML) models, the data generated based at least in part on the notation(s) of the medical provider, and the data generated based at least in part on the interaction(s) between the patient and the physical infrastructure of the medical organization, one or more standardized billing codes associated with the examination of the patient.

A further example aspect of the present disclosure is directed to one or more non-transitory computer-readable media. The non-transitory computer-readable media may comprise instructions that when executed by one or more computing devices cause the computing device(s) to perform operations. The operations may include receiving data associated with one or more patients and describing at least one of: a plurality of admission notes and associated standardized billing codes, or a plurality of subjective objective assessment and plan (SOAP) notes and associated standardized billing codes. The operations may also include receiving data describing one or more interactions between the patient(s) and physical infrastructure of one or more medical organizations that evaluated the patient(s). The operations may further include generating, based at least in part on the data associated with the patient(s) and the data describing the interaction(s) between the patient(s) and the physical infrastructure of the medical organization(s) that evaluated the patient(s), one or more machine learning (ML) models configured to determine one or more standardized billing codes associated with an examination of a patient by a medical provider associated with at least one of the medical organization(s).

Other aspects of the present disclosure are directed to various systems, apparatuses, non-transitory computer-readable media, user interfaces, and electronic devices.

These and other features, aspects, and advantages of various embodiments of the present disclosure will be better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in this specification, which makes reference to the appended figures, in which:

FIG. 1 depicts an example computing environment according to example embodiments of the present disclosure;

FIGS. 2A-C depict an example event sequence according to example embodiments of the present disclosure;

FIGS. 3A, 3B, 4A, 4B, 5A, 5B, 6A, and 6B depict example interfaces according to example embodiments of the present disclosure; and

FIGS. 7 and 8 depict example methods according to example embodiments of the present disclosure.

### DETAILED DESCRIPTION

With reference now to the Figures, example embodiments of the present disclosure will be discussed in further detail.

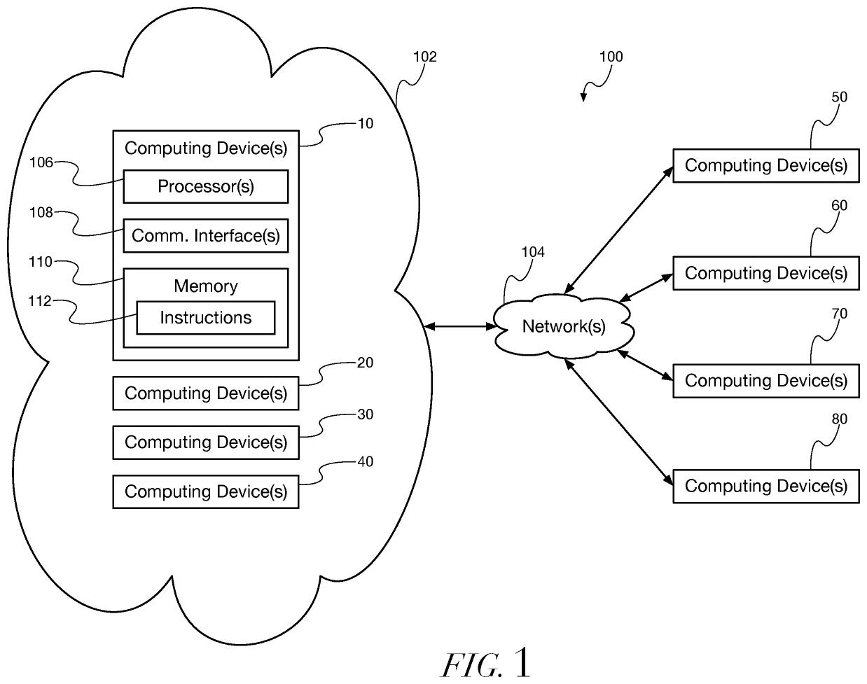

FIG. 1 depicts an example computing environment according to example embodiments of the present disclosure.

Referring to FIG. 1, environment 100 may include one or more computing devices (e.g., one or more desktop computers, laptop computers, set-top devices, tablet computers, mobile devices, smartphones, wearable devices, servers, and/or the like). For example, environment 100 may include computing devices 10, 20, 30, 40, 50, 60, 70, and/or 80, any one of which may include one or more associated and/or component computing devices (e.g., a mobile device and an associated wearable device, and/or the like). Environment 100 may also include one or more networks, for example, network(s) 102 and/or 104 (e.g., one or more wired networks, wireless networks, and/or the like). Network(s) 102 may interface computing device(s) 10, 20, 30, and/or 40, with one another and/or computing device(s) 50, 60, 70, and/or 80 (e.g., via network(s) 104, and/or the like).

Computing device(s) 10 may include one or more processor(s) 106, one or more communication interfaces 108, and memory 110 (e.g., one or more hardware components for storing executable instructions, data, and/or the like). Communication interface(s) 108 may enable computing device(s) 10 to communicate with computing device(s) 20, 30, 40, 50, 60, 70, and/or 80 (e.g., via network(s) 102, 104, and/or the like). Memory 110 may include (e.g., store, and/or the like) instructions 112. When executed by processor(s) 106, instructions 112 may cause computing device(s) 10 to perform one or more operations, functions, and/or the like described herein. It will be appreciated that computing device(s) 20, 30, 40, 50, 60, 70, and/or 80 may include one or more of the components described above with respect to computing device(s) 10.

Unless explicitly indicated otherwise, the operations, functions, and/or the like described herein may be performed by computing device(s) 10, 20, 30, 40, 50, 60, 70, and/or 80 (e.g., by computing device(s) 10, 20, 30, 40, 50, 60, 70, or 80, by any combination of one or more of computing device(s) 10, 20, 30, 40, 50, 60, 70, and/or 80, and/or the like).

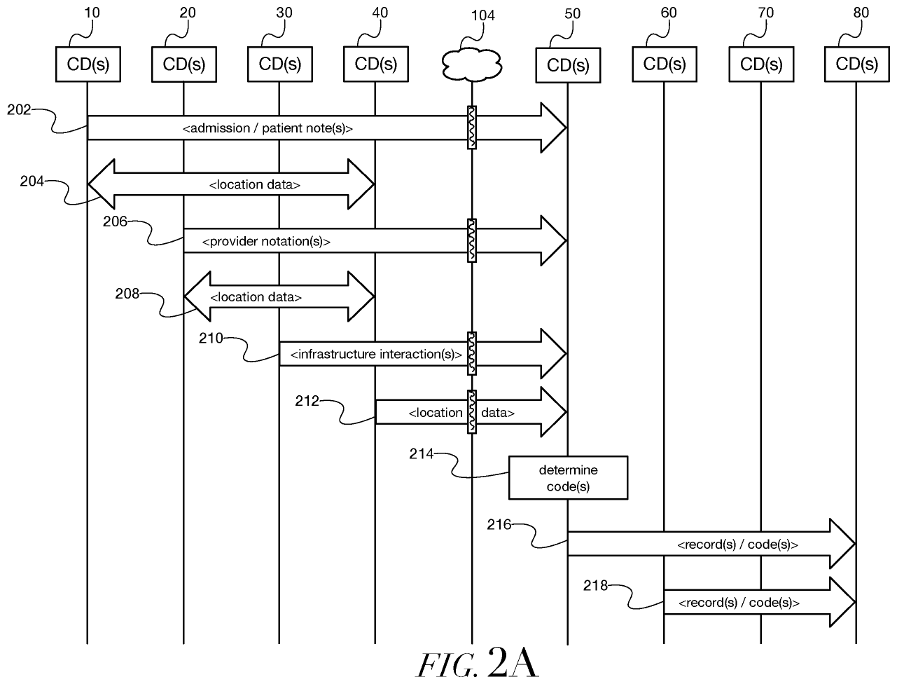

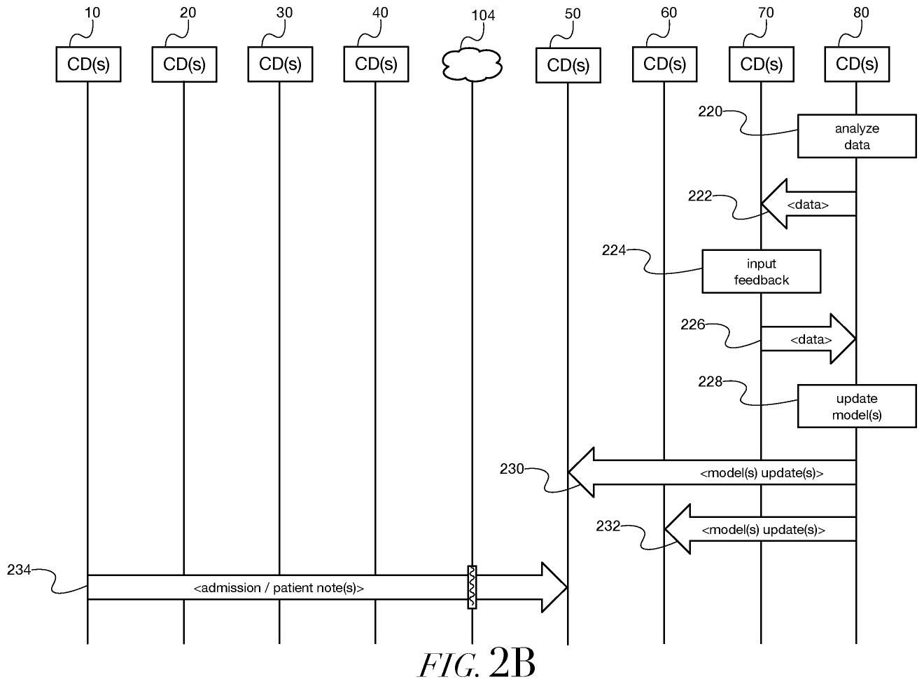

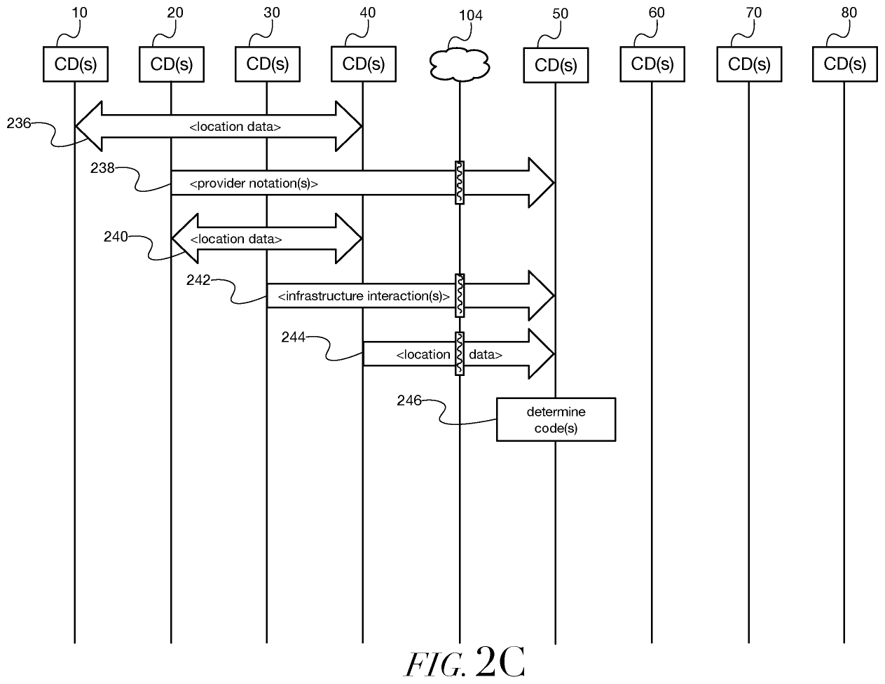

FIGS. 2A-C depict an example event sequence according to example embodiments of the present disclosure.

Referring to FIG. 2A, at (202), computing device(s) 10 may communicate (e.g., determine, generate, transmit, and/or the like), to computing device(s) 50 (e.g., via network(s) 104 (as indicated by the pattern-filled box over the line extending downward from network(s) 104), and/or the like), data based at least in part on one or more admission, intake, and/or the like notes associated with a patient (e.g., provided by the patient, one or more admission personnel associated with a medical organization, and/or the like), and computing device(s) 50 (e.g., one or more servers, and/or the like) may receive such data. The admission note(s) may indicate, for example, the reason for the patient's visit, appointment, and/or the like, as well as other comments (e.g., regarding observed symptoms, complaints, and/or the like).

In some embodiments, the admission note(s) may include insurance information for the patient, which may be verified (e.g., for preauthorization, to determine whether coverage is in network, out of network, and/or the like), for example, by computing device(s) 10, 50, and/or the like.

In some embodiments, the admission note(s) may be provided via one or more computing devices (e.g., one or more of computing device(s) 10, and/or the like) located within one or more physically secured enclosures housing one or more of the patient, admission personnel, and/or the like. For example, such enclosure(s) may be configured, designed, and/or the like in accordance with one or more aspects of U.S. Pat. No. 9,963,892, issued May 8, 2018, and entitled “MODULAR PRIVACY BOOTH FOR COOPERATIVE USE WITH A TELLER STATION, ATM, OR THE LIKE,” the disclosure of which is incorporated by reference herein in its entirety. Additionally or alternatively, the admission note(s) may be provided via one or more personal mobile devices (e.g., one or more of computing device(s) 10, and/or the like) associated with the patient, admission personnel, medical organization, and/or the like. For example, one or more portions of the admission note(s) may be provided via text message, a website, a social network application, an insurance application, and/or the like. In some embodiments, one or more portions of the admission note(s) may be provided via one or more applications associated with the medical organization and executing on one or more of such personal mobile device(s), and/or the like.

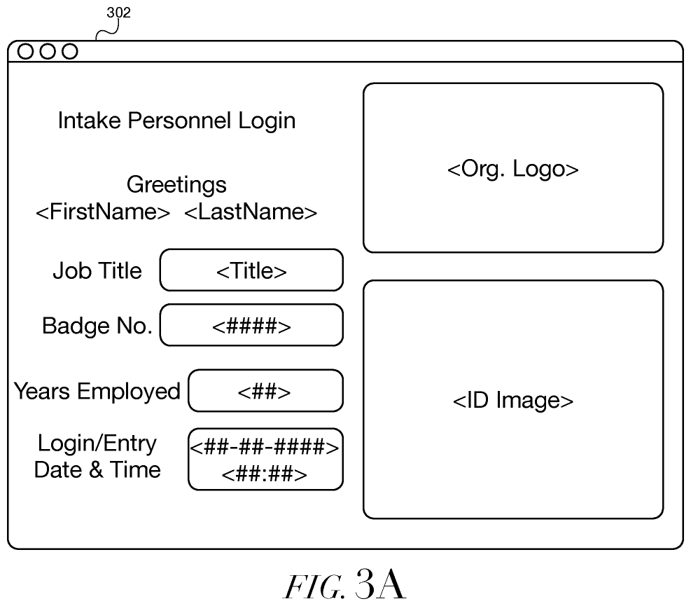

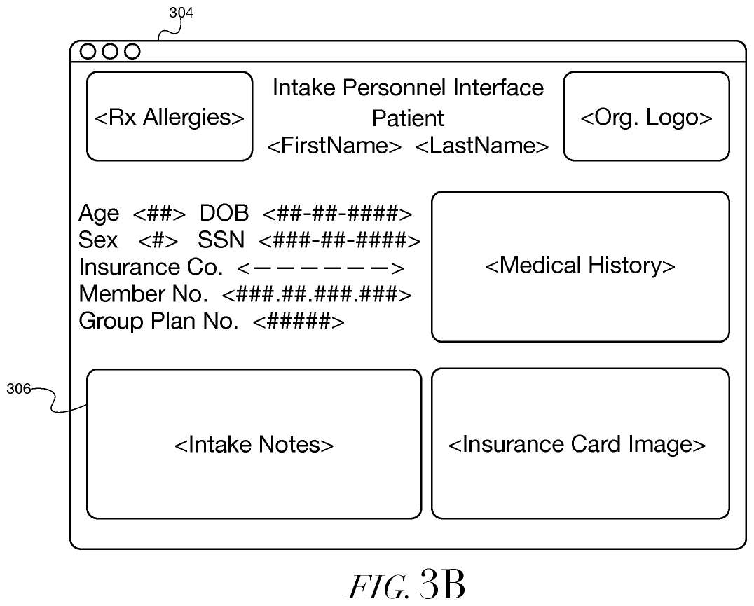

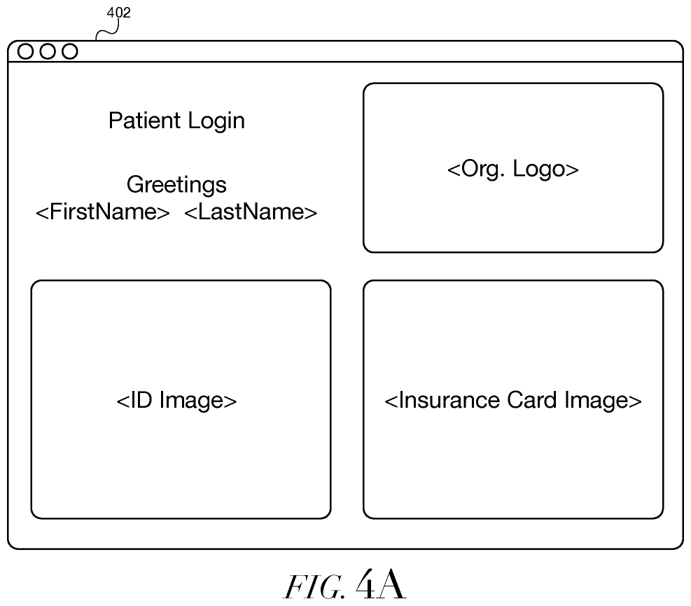

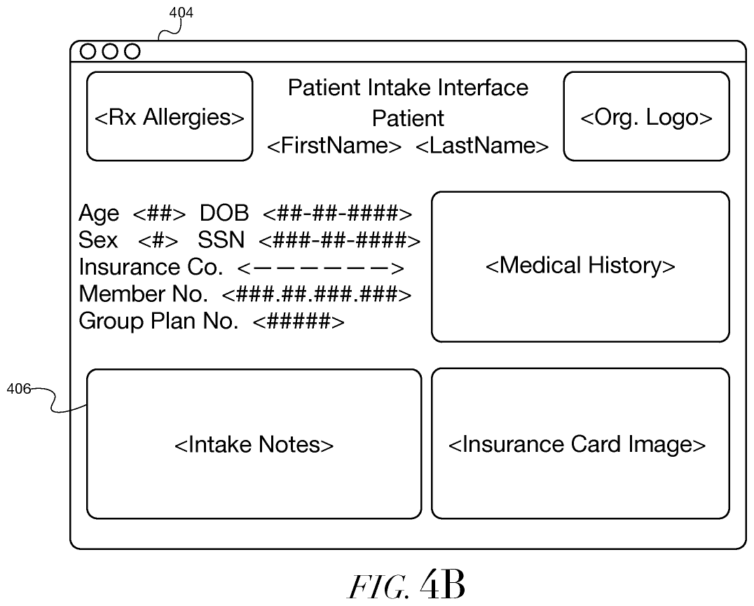

In some embodiments, one or more interfaces may be provided to the patient, one or more of the admission personnel, and/or the like for authentication prior to entry, communication, and/or the like of the admission note(s). For example, referring to FIGS. 3A and 4A, interfaces 302, 402, and/or the like may be provided to the patient, one or more of the admission personnel, and/or the like for authentication (e.g., via password, passcode, credentials, physical security devices, biometrics, and/or the like). Responsive to authentication, one or more subsequent interfaces may be provided to the patient, one or more of the admission personnel, and/or the like for entry of the admission note(s), and/or the like. For example, referring to FIGS. 3B and 4B, interfaces 304, 404, and/or the like may be provided to the patient, one or more of the admission personnel, and/or the like. As illustrated, interfaces 304 and 404 may include one or more elements 306, 406, and/or the like for entry of the admission note(s), and/or the like.

Returning to FIG. 2A, at (204), computing device(s) 10 and computing device(s) 40 may communicate (e.g., determine, generate, transmit, receive, and/or the like) data based at least in part on one or more locations of the patient, one or more of the admission personnel, and/or the like within physical infrastructure of the medical organization (e.g., based at least in part on data generated, received, and/or the like by one or more mobile devices, cameras, sensors, tags, and/or the like associated with the patient, admission personnel, one or more portions of the physical infrastructure, and/or the like). In some embodiments, the data may comprise data generated responsive to communication between the physical infrastructure of the medical organization and one or more mobile devices (e.g., computing device(s) 10, and/or the like) associated with the patient, one or more of the admission personnel, and/or the like. In some of such embodiments, the data may be generated at least in part by an application associated with the medical organization and executing on one or more of such personal mobile device(s), and/or the like.

At (206), computing device(s) 20 may communicate, to computing device(s) 50, data based at least in part on one or more notations of a medical provider with respect to an examination of the patient, and computing device(s) 50 may receive such data. As used herein, “examination of a patient” includes any interaction (e.g., physical, virtual, direct, indirect, and/or the like) in which a patient is assessed by a medical provider (e.g., doctor, nurse, physician assistant, nurse practitioner, skilled, licensed, or authorized practitioner, therapist, and/or the like), including, for example, physical evaluation, psychiatric evaluation, psychological evaluation, and/or the like, as well as associated activities, e.g., tests, procedures, surgeries, interventions, therapies, imagery, lab work, analyses, and/or the like. In some embodiments the notation(s) may include one or more subjective objective assessment and plan (SOAP) notes.

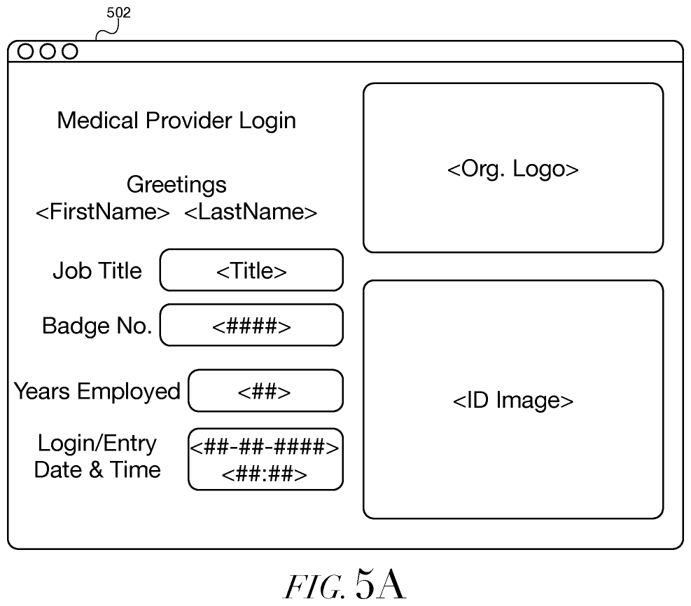

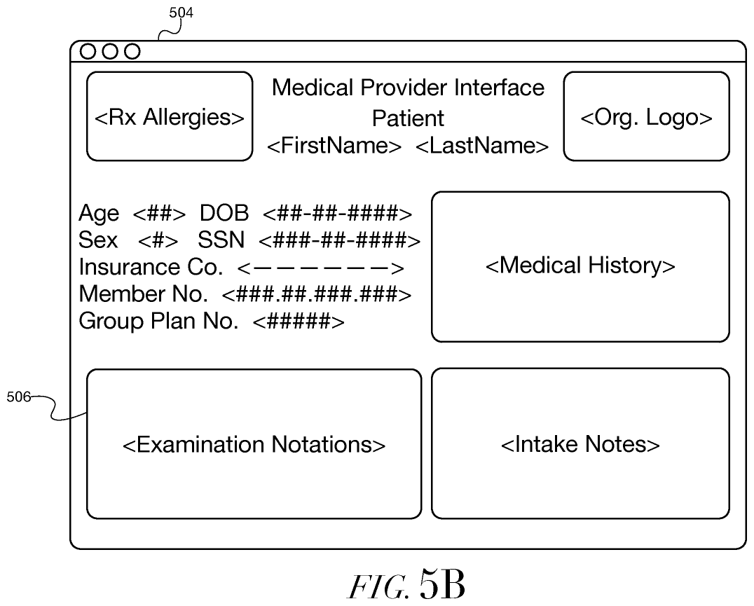

In some embodiments, one or more interfaces may be provided to the medical provider for authentication prior to entry, communication, and/or the like of the notation(s). For example, referring to FIG. 5A, interface 502, and/or the like may be provided to the medical provider for authentication, and/or the like. Responsive to authentication, one or more subsequent interfaces may be provided to the medical provider for entry of the notation(s), and/or the like. For example, referring to FIG. 5B, interface 504, and/or the like may be provided to the medical provider. As illustrated, interface 504 may include one or more elements 506, and/or the like for entry of the notation(s), and/or the like.

Returning to FIG. 2A, at (208), computing device(s) 20 and computing device(s) 40 may communicate data based at least in part on one or more locations of the patient, medical provider, and/or the like within the physical infrastructure of the medical organization (e.g., based at least in part on data generated, received, and/or the like by one or more mobile devices, cameras, sensors, tags, and/or the like associated with the patient, medical provider, one or more portions of the physical infrastructure, and/or the like). In some embodiments, the data may comprise data generated responsive to communication between the physical infrastructure of the medical organization and one or more mobile devices (e.g., computing device(s) 10, 20, and/or the like) associated with the patient, medical provider, and/or the like. In some of such embodiments, the data may be generated at least in part by an application associated with the medical organization and executing on one or more of such personal mobile device(s), and/or the like.

At (210), computing device(s) 30 may communicate, to computing device(s) 50, data generated based at least in part on one or more interactions between the patient and the physical infrastructure of the medical organization, and computing device(s) 50 may receive such data. For example, in some embodiments, the data may comprise one or more medical images associated with the patient, one or more lab reports associated with the patient, and/or the like.

At (212), computing device(s) 40 may communicate, to computing device(s) 50, data based at least in part on one or more determined (e.g., based at least in part on the data communicated at (204), (208), and/or the like) locations (e.g., within the physical infrastructure of the medical organization, and/or the like) for one or more of the admission personnel, patient, medical provider, and/or the like, and computing device(s) 50 may receive such data. For example, the data may comprise one or more location-based timestamps associated with one or more of the admission personnel, patient, medical provider, and/or the like (e.g., indicating their respective locations within the physical infrastructure of the medical organization at various times, and/or the like).

At (214), computing device(s) 50 (e.g., one or more servers associated with the medical organization, and/or the like) may determine (e.g., based at least in part on the data communicated at (202), (206), (210), (212), and/or the like) one or more standardized billing codes associated with the examination of the patient (e.g., associated with one or more principal diagnoses, and/or the like). For example, computing device(s) 50 may determine such billing code(s) based at least in part on the admission note(s), notation(s) of the medical provider (e.g., SOAP notes, and/or the like), data generated based at least in part on the interaction(s) between the patient and the physical infrastructure of the medical organization, and/or the like.

In some embodiments, determining one or more of the billing code(s) may comprise parsing one or more of the admission note(s), notation(s) of the medical provider, and/or the like, for example, to identify one or more predetermined terms, phrases, and/or the like associated with one or more of the billing code(s), and/or the like. Additionally or alternatively, determining one or more of the billing code(s) may comprise analyzing one or more of the medical image(s), lab report(s), and/or the like associated with the patient. In some embodiments, determining one or more of the billing code(s) may comprise determining (e.g., based at least in part on one or more location-based timestamps associated with the patient, medical provider, and/or the like) an amount of time spent by the medical provider with the patient (e.g., within one or more portions of the physical infrastructure of the medical organization, and/or the like).

In some embodiments, determining one or more of the billing code(s) may comprise determining one or more of the billing code(s) based at least in part on one or more machine learning (ML) models (e.g., one or more neural networks, and/or the like). In some of such embodiments, one or more of such ML model(s) may have been generated (e.g., by computing device(s) 50, and/or the like) based at least in part on a corpus (e.g., training data, and/or the like) comprising admission notes and associated standardized billing codes, notations of medical providers (e.g., SOAP notes, and/or the like) and associated standardized billing codes, and/or the like. Additionally or alternatively, one or more of the ML model(s) may have been generated based at least in part on data describing one or more medical histories of one or more patients (e.g., associated with the admission notes, notations of the medical providers, and/or the like), one or more interactions between such patient(s) and physical infrastructure of one or more medical organizations that evaluated the patient(s), and/or the like.

In some embodiments, determining one or more of the billing code(s) may comprise verifying that such code(s) are approved, accepted, and/or the like by the patient's insurance, whether such code(s) are in network, out of network, and/or the like with the patient's insurance, and/or the like.

At (216), computing device(s) 50 may communicate, to computing device(s) 80 (e.g., one or more servers associated with aggregate data storage, processing, analysis, and/or the like) data indicating the one or more determined billing code(s), describing associated admission note(s), notation(s) of the medical provider, data generated based at least in part on interactions between the patient and the physical infrastructure of the medical organization, medical histories, records, and/or the like, and computing device(s) 80 may receive such data.

Similarly, at (218), computing device(s) 60 (e.g., one or more servers associated with one or more different medical organizations, locations, and/or the like) may communicate analogous data (e.g., associated with distinct examinations of different patients, and/or the like) to computing device(s) 80, which may receive such data.

Referring to FIG. 2B, at (220), computing device(s) 80 may analyze the received data (e.g., communicated at (216), (218), and/or the like), for example, to identify one or more determined billing codes for review by audit personnel, and/or the like. In some embodiments, computing device(s) 80 may analyze, mine, and/or the like such data to identify potential fraud, identity theft, one or more trends, and/or the like. For example, such data may be analyzed to determine one or more trends based on populations, diagnoses, geographic information, demographic information, time of day, insurance type, and/or the like. Potential fraud may include, for example, upcoding, unbundling, coding for higher reimbursements than were actually performed, billing bundled procedures separately for services included in a global fee, inappropriate use of modifier codes, coding for services that were medically unnecessary, offered limited value, and/or the like. In some of such embodiments, one or more aspects of the data (e.g., patient-identifying information, and/or the like) may be anonymized, and/or the like. In some embodiments, computing device(s) 80 may analyze, mine, and/or the like the data to determine the effectiveness of employing one or more physically secured enclosures housing one or more of the patient, admission personnel, and/or the like.

At (222), computing device(s) 80 may communicate, to computing device(s) 70 (e.g., associated with one or more audit personnel, and/or the like), data indicating the determined billing code(s) for review, describing associated admission note(s), notation(s) of the medical provider(s), data generated based at least in part on interactions between the associated patient(s) and the physical infrastructure of the medical organization(s), medical histories, records, and/or the like, and computing device(s) 70 may receive such data.

At (224), computing device(s) 70 may receive input (e.g., from the audit personnel, and/or the like) modifying one or more of the determined billing code(s), for example, based at least in part on a review (e.g., a manual review, and/or the like) of the determined billing code(s), associated admission note(s), notation(s) of the medical provider, data based at least in part on the interactions between the associated patient(s) and the physical infrastructure of the medical organization(s), medical histories, records, and/or the like.

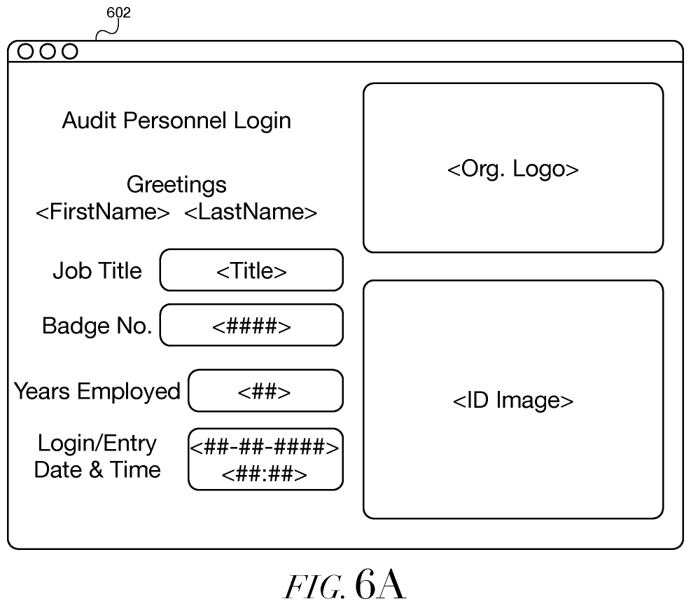

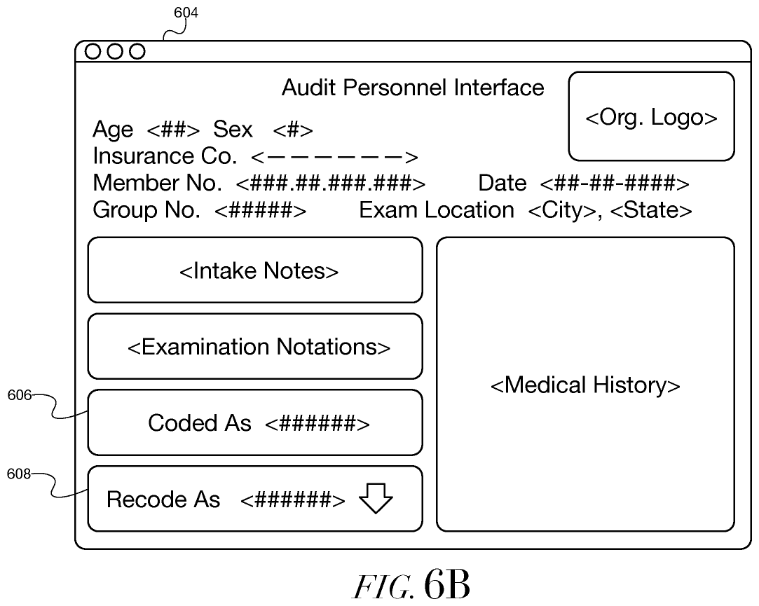

In some embodiments, one or more interfaces may be provided to the audit personnel for authentication prior to modification of one or more of the determined billing code(s). For example, referring to FIG. 6A, interface 602, and/or the like may be provided to the audit personnel for authentication, and/or the like. Responsive to authentication, one or more subsequent interfaces may be provided to the audit personnel for modifying such determined billing code(s), and/or the like. For example, referring to FIG. 6B, interface 604, and/or the like may be provided to the audit personnel. As illustrated, interface 604 may include one or more elements 606, and/or the like for indicating the determined billing code(s), and one or more elements 608 for modifying such billing code(s), and/or the like.

Returning to FIG. 2B, at (226), computing device(s) 70 may communicate, to computing device(s) 80, data indicating the one or more modifications (e.g., based at least in part on the audit, and/or the like) to the one or more of the billing code(s), and/or the like, and computing device(s) 80 may receive such data.

At (228), computing device(s) 80 may update (e.g., modify, generate, and/or the like) one or more of the ML model(s) based at least in part on the modification(s) to the billing code(s), and/or the like.

At (230), computing device(s) 80 may communicate, to computing device(s) 50, data based at least in part on the updated ML model(s) (e.g., data describing the updated ML model(s), data indicating one or more modifications for incorporation into one or more existing ML models, and/or the like), and computing device(s) 50 may receive such data and update one or more of its ML model(s) in accordance therewith, and/or the like.

Similarly, at (232), computing device(s) 80 may communicate, to computing device(s) 60, data based at least in part on the updated ML model(s), and computing device(s) 60 may receive such data and update one or more of its ML model(s) in accordance therewith, and/or the like.

At (234), computing device(s) 10 may communicate, to computing device(s) 50, data based at least in part on one or more admission, intake, and/or the like notes associated with a different patient, and computing device(s) 50 may receive such data.

Referring to FIG. 2C, at (236), computing device(s) 10 and computing device(s) 40 may communicate data based at least in part on one or more locations of the different patient, one or more of the admission personnel, and/or the like within physical infrastructure of the medical organization.

At (238), computing device(s) 20 may communicate, to computing device(s) 50, data based at least in part on one or more notations of a different medical provider with respect to an examination of the different patient, and computing device(s) 50 may receive such data.

At (240), computing device(s) 20 and computing device(s) 40 may communicate data based at least in part on one or more locations of the different patient, different medical provider, and/or the like within the physical infrastructure of the medical organization.

At (242), computing device(s) 30 may communicate, to computing device(s) 50, data generated based at least in part on one or more interactions between the different patient and the physical infrastructure of the medical organization, and computing device(s) 50 may receive such data.

At (244), computing device(s) 40 may communicate, to computing device(s) 50, data based at least in part on one or more determined (e.g., based at least in part on the data communicated at (236), (240), and/or the like) locations (e.g., within the physical infrastructure of the medical organization, and/or the like) for one or more of the admission personnel, different patient, different medical provider, and/or the like, and computing device(s) 50 may receive such data.

At (246), computing device(s) 50 may determine (e.g., based at least in part on the data communicated at (234), (238), (242), (244), and/or the like) one or more standardized billing codes associated with the examination of the different patient. For example, in some embodiments, computing device(s) 50 may determine one or more of such billing code(s) based at least in part on one or more of the updated ML model(s).

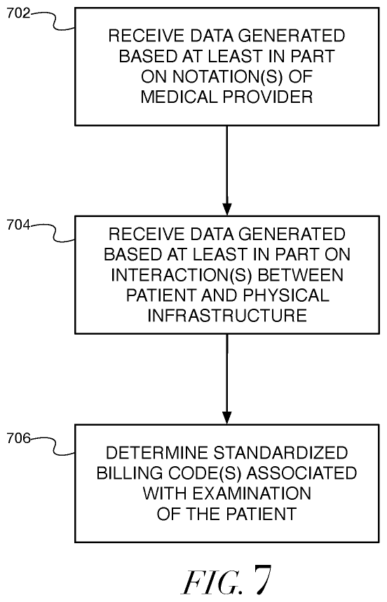

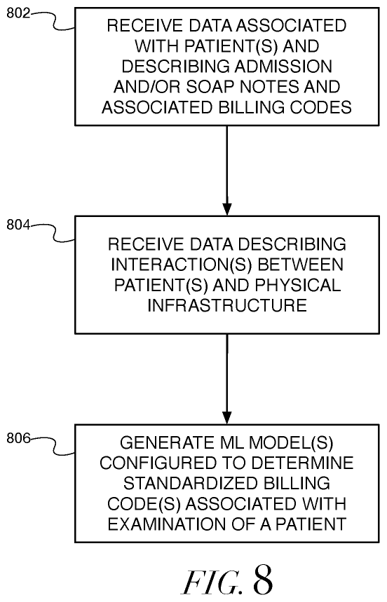

FIGS. 7 and 8 depict example methods according to example embodiments of the present disclosure.

Referring to FIG. 7, at (702), one or more computing devices may receive data generated based at least in part on one or more notations of a medical provider with respect to an examination of a patient. For example, computing device(s) 50 may receive (e.g., from computing device(s) 20, and/or the like) data generated based at least in part on one or more notations of a medical provider with respect to an examination of a patient.

At (704), the computing device(s) may receive data generated based at least in part on one or more interactions between the patient and physical infrastructure of a medical organization associated with the medical provider. For example, computing device(s) 50 may receive (e.g., from computing device(s) 30, 40, and/or the like) data generated based at least in part on one or more interactions between the patient and physical infrastructure of a medical organization associated with the medical provider.

At (706), the computing device(s) may determine, based at least in part on the data generated based at least in part on the notation(s) of the medical provider and the data generated based at least in part on the interaction(s) between the patient and the physical infrastructure of the medical organization, one or more standardized billing codes associated with the examination of the patient. For example, computing device(s) 50 may determine (e.g., based at least in part on the data received from computing device(s) 10, 20, 30, 40, and/or the like) one or more standardized billing codes associated with the examination of the patient.

Referring to FIG. 8, at (802), one or more computing devices may receive data associated with one or more patients. Such data may describe a plurality of admission notes and associated standardized billing codes, a plurality of notations of medical providers (e.g., SOAP notes, and/or the like) and associated standardized billing codes, and/or the like. For example, computing device(s) 80 may receive such data (e.g., from computing device(s) 50, 60, and/or the like).

At (804), the computing device(s) may receive data describing one or more interactions between the patient(s) and physical infrastructure of one or more medical organizations that evaluated the patient(s). For example, computing device(s) 80 may receive such data (e.g., from computing device(s) 50, 60, and/or the like).

At (806), the computing device(s) may generate (e.g., based at least in part on the received data, and/or the like) one or more ML models configured to determine one or more standardized billing codes associated with an examination of a patient by a medical provider associated with at least one of the medical organization(s). For example, computing device(s) 80 may generate (e.g., based at least in part on the data received from computing device(s) 50, 60, and/or the like) one or more ML models (e.g., new ML model(s), updated ML model(s), and/or the like) configured to determine one or more standardized billing codes associated with an examination of a patient by a medical provider associated with at least one of the medical organization(s).

The technology discussed herein makes reference to servers, databases, software applications, and/or other computer-based systems, as well as actions taken and information sent to and/or from such systems. The inherent flexibility of computer-based systems allows for a great variety of possible configurations, combinations, and/or divisions of tasks and/or functionality between and/or among components. For instance, processes discussed herein may be implemented using a single device or component and/or multiple devices or components working in combination. Databases and/or applications may be implemented on a single system and/or distributed across multiple systems. Distributed components may operate sequentially and/or in parallel.

Various connections between elements are discussed in the above description. These connections are general and, unless specified otherwise, may be direct and/or indirect, wired and/or wireless. In this respect, the specification is not intended to be limiting.

The depicted and/or described steps are merely illustrative and may be omitted, combined, and/or performed in an order other than that depicted and/or described; the numbering of depicted steps is merely for ease of reference and does not imply any particular ordering is necessary or preferred.

The functions and/or steps described herein may be embodied in computer-usable data and/or computer-executable instructions, executed by one or more computers and/or other devices to perform one or more functions described herein. Generally, such data and/or instructions include routines, programs, objects, components, data structures, or the like that perform particular tasks and/or implement particular data types when executed by one or more processors of a computer and/or other data-processing device. The computer-executable instructions may be stored on a computer-readable medium such as a hard disk, optical disk, removable storage media, solid-state memory, read-only memory (ROM), random-access memory (RAM), or the like. As will be appreciated, the functionality of such instructions may be combined and/or distributed as desired. In addition, the functionality may be embodied in whole or in part in firmware and/or hardware equivalents, such as integrated circuits, application-specific integrated circuits (ASICs), field-programmable gate arrays (FPGAs), or the like. Particular data structures may be used to more effectively implement one or more aspects of the disclosure, and such data structures are contemplated to be within the scope of computer-executable instructions and/or computer-usable data described herein.

Although not required, one of ordinary skill in the art will appreciate that various aspects described herein may be embodied as a method, system, apparatus, and/or one or more computer-readable media storing computer-executable instructions. Accordingly, aspects may take the form of an entirely hardware embodiment, an entirely software embodiment, an entirely firmware embodiment, and/or an embodiment combining software, hardware, and/or firmware aspects in any combination.

As described herein, the various methods and acts may be operative across one or more computing devices and/or networks. The functionality may be distributed in any manner or may be located in a single computing device (e.g., server, client computer, user device, or the like).

Aspects of the disclosure have been described in terms of illustrative embodiments thereof. Numerous other embodiments, modifications, and/or variations within the scope and spirit of the appended claims may occur to persons of ordinary skill in the art from a review of this disclosure. For example, one of ordinary skill in the art may appreciate that the steps depicted and/or described may be performed in other than the recited order and/or that one or more illustrated steps may be optional and/or combined. Any and all features in the following claims may be combined and/or rearranged in any way possible.

While the present subject matter has been described in detail with respect to various specific example embodiments thereof, each example is provided by way of explanation, not limitation of the disclosure. Those skilled in the art, upon attaining an understanding of the foregoing, may readily produce alterations to, variations of, and/or equivalents to such embodiments. Accordingly, the subject disclosure does not preclude inclusion of such modifications, variations, and/or additions to the present subject matter as would be readily apparent to one of ordinary skill in the art. For instance, features illustrated and/or described as part of one embodiment may be used with another embodiment to yield a still further embodiment. Thus, it is intended that the present disclosure cover such alterations, variations, and/or equivalents.

What is claimed is:

1. A method comprising:
receiving, by one or more computing devices, data generated based at least in part on one or more notations of a medical provider with respect to an examination of a patient;
receiving, by the one or more computing devices, data generated based at least in part on one or more interactions between the patient and physical infrastructure of a medical organization associated with the medical provider; and
determining, by the one or more computing devices and based at least in part on the data generated based at least in part on the one or more notations of the medical provider and the data generated based at least in part on the one or more interactions between the patient and the physical infrastructure of the medical organization, one or more standardized billing codes associated with the examination of the patient.

2. The method of claim 1, wherein:
the one or more notations of the medical provider comprise one or more subjective objective assessment and plan (SOAP) notes; and
determining the one or more standardized billing codes associated with the examination comprises parsing the one or more SOAP notes to identify one or more predetermined terms or phrases associated with the one or more standardized billing codes.

3. The method of claim 1, wherein determining the one or more standardized billing codes associated with the examination comprises determining the one or more standardized billing codes based at least in part on one or more admission notes provided by at least one of the patient or one or more admission personnel associated with the medical organization.

4. The method of claim 3, wherein the one or more admission notes are provided by the patient via an application associated with the medical organization and executing on a personal mobile device associated with the patient.

5. The method of claim 3, wherein the one or more admission notes are provided by the at least one of the patient or the one or more admission personnel via one or more computing devices located within a physically secured enclosure housing the at least one of the patient or the one or more admission personnel.

6. The method of claim 1, wherein determining the one or more standardized billing codes associated with the examination comprises determining the one or more standardized billing codes based at least in part on one or more machine learning (ML) models generated based at least in part on a corpus of at least one of:
a plurality of admission notes and associated standardized billing codes; or
a plurality of subjective objective assessment and plan (SOAP) notes and associated standardized billing codes.

7. The method of claim 6, wherein the one or more ML models are generated based at least in part on data describing:
one or more medical histories of one or more patients associated with:
one or more of the plurality of admission notes, or
one or more of the plurality of SOAP notes; and
one or more interactions between the one or more patients and physical infrastructure of one or more medical organizations that evaluated the one or more patients.

8. The method of claim 6, comprising receiving, by the one or more computing devices, data indicating one or more modifications to the standardized billing codes associated with the examination, the one or more modifications being associated with an audit based at least in part on:
the data generated based at least in part on the one or more notations of the medical provider, or
the data generated based at least in part on the one or more interactions between the patient and the physical infrastructure of the medical organization.

9. The method of claim 8, comprising generating, by the one or more computing devices and based at least in part on the one or more modifications to the standardized billing codes associated with the audit, one or more updated ML models.

10. The method of claim 9, comprising:
receiving, by the one or more computing devices, data generated based at least in part on one or more notations of a different medical provider with respect to an examination of a different patient;
receiving, by the one or more computing devices, data generated based at least in part on one or more interactions between the different patient and physical infrastructure of a medical organization associated with the different medical provider; and
determining, by the one or more computing devices and based at least in part on the one or more updated ML models, the data generated based at least in part on the one or more notations of the different medical provider, and the data generated based at least in part on the one or more interactions between the different patient and the physical infrastructure of the medical organization associated with the different medical provider, one or more standardized billing codes associated with the examination of the different patient.

11. The method of claim 1, wherein:
the data generated based at least in part on the one or more interactions between the patient and the physical infrastructure comprises at least one of:
one or more medical images associated with the patient, or
one or more lab reports associated with the patient; and
determining the one or more standardized billing codes comprises determining the one or more standardized billing codes based at least in part on analyzing the at least one of the one or more medical images associated with the patient or the one or more lab reports associated with the patient.

12. The method of claim 1, wherein determining the one or more standardized billing codes associated with the examination comprises determining that the one or more standardized billing codes are at least one of:
approved by an insurance provider of the patient;
accepted by an insurance provider of the patient; or
in network for an insurance provider of the patient.

13. The method of claim 1, wherein:
the data generated based at least in part on the one or more interactions between the patient and the physical infrastructure comprises one or more location-based timestamps associated with the patient and one or more location-based timestamps associated with the medical provider; and
determining the one or more standardized billing codes comprises determining, based at least in part on the one or more location-based timestamps associated with the patient and the one or more location-based timestamps associated with the medical provider, an amount of time spent by the medical provider with the patient within at least a portion of the physical infrastructure.

14. The method of claim 1, wherein:
the data generated based at least in part on the one or more interactions between the patient and the physical infrastructure comprises data generated responsive to communication between the physical infrastructure and a mobile device associated with at least one of the patient or the medical provider; and
determining the one or more standardized billing codes comprises determining the one or more standardized billings codes based at least in part on the data generated responsive to the communication between the physical infrastructure and the mobile device associated with the at least one of the patient or the medical provider.

15. The method of claim 14, wherein:
the data generated responsive to the communication between the physical infrastructure and the mobile device comprises data generated at least in part by an application associated with the medical organization and executing on the mobile device; and
determining the one or more standardized billing codes comprises determining the one or more standardized billings codes based at least in part on the data generated at least in part by the application associated with the medical organization and executing on the mobile device.

16. A system comprising:
one or more processors; and
a memory storing instructions that when executed by the one or more processors cause the system to perform operations comprising:
receiving data generated based at least in part on one or more notations of a medical provider with respect to an examination of a patient;
receiving data generated based at least in part on one or more interactions between the patient and physical infrastructure of a medical organization associated with the medical provider; and
determining, based at least in part on one or more machine learning (ML) models, the data generated based at least in part on the one or more notations of the medical provider, and the data generated based at least in part on the one or more interactions between the patient and the physical infrastructure of the medical organization, one or more standardized billing codes associated with the examination of the patient.

17. The system of claim 16, wherein the operations comprise generating the one or more ML models based at least in part on a corpus of at least one of:
a plurality of admission notes and associated standardized billing codes; or
a plurality of subjective objective assessment and plan (SOAP) notes and associated standardized billing codes.

18. The system of claim 17, wherein the operations comprise generating the one or more ML models based at least in part on data describing:
one or more medical histories of one or more patients associated with:
one or more of the plurality of admission notes, or
one or more of the plurality of SOAP notes; and
one or more interactions between the one or more patients and physical infrastructure of one or more medical organizations that evaluated the one or more patients.

19. The system of claim 16, wherein the operations comprise:
receiving data indicating one or more modifications to the standardized billing codes associated with an audit based at least in part on:
the data generated based at least in part on the one or more notations of the medical provider, or
the data generated based at least in part on the one or more interactions between the patient and the physical infrastructure of the medical organization; and
generating, based at least in part on the one or more modifications to the standardized billing codes associated with the audit, one or more updated ML models.

20. One or more non-transitory computer-readable media comprising instructions that when executed by one or more computing devices cause the one or more computing devices to perform operations comprising:
receiving data associated with one or more patients and describing at least one of:
a plurality of admission notes and associated standardized billing codes, or
a plurality of subjective objective assessment and plan (SOAP) notes and associated standardized billing codes;
receiving data describing one or more interactions between the one or more patients and physical infrastructure of one or more medical organizations that evaluated the one or more patients; and
generating, based at least in part on the data associated with the one or more patients and the data describing the one or more interactions between the one or more patients and the physical infrastructure of the one or more medical organizations that evaluated the one or more patients, one or more machine learning (ML) models configured to determine one or more standardized billing codes associated with an examination of a patient by a medical provider associated with at least one of the one or more medical organizations.
